(19) United States
(12) Patent Application Publication
(10) Pub. No.: US 2025/0092365 A1
(43) Pub. Date: Mar. 20, 2025

(54) METHOD OF MANUFACTURING AUTOLOGOUS CARDIAC LINEAGE CELLS

(71) Applicant: ReGen Theranostics, Inc., Rochester, MN (US)

(72) Inventor: Timothy Nelson, Rochester, MN (US)

(73) Assignee: ReGen Theranostics, Inc., Rochester, MN (US)

(21) Appl. No.: 18/368,240

(22) Filed: Sep. 14, 2023

(51) Int. Cl. C12N 5/077; A61K 35/34; C12N 15/86

(52) U.S. Cl. CPC C12N 5/0657; A61K 35/34; C12N 15/86; C12N 2501/415; C12N 2506/1307; C12N 2760/18843

(57) ABSTRACT

A method of manufacturing autologous cardiac lineage cells, the method includes receiving a patient-specific sample from a subject, producing a plurality of fibroblast cells as a function of the patient-specific sample, wherein producing the plurality of fibroblast cells includes transferring the patient-specific sample to a first growth media, generating a plurality of induced pluripotent stem cells (iPSCs) as a function of the plurality of fibroblast cells, wherein generating the plurality of iPSCs includes identifying a plurality of confluent iPSCs from the plurality of iPSCs, and differentiating the plurality of iPSCs into a plurality of cardiac lineage cells, wherein differentiating the plurality of iPSCs into a plurality of cardiac lineage cells includes performing a two-dimensional (2D) expansion process on the plurality of confluent iPSCs and performing a three-dimensional (3D) expansion process on the plurality of 2D-expended confluent iPSCs.

### FIELD OF THE INVENTION

The present invention generally relates to the field of regenerative medicine. In particular, the present invention is directed to a method of manufacturing autologous cardiac lineage cells.

### BACKGROUND

The heart is a vital organ that pumps blood and oxygen to the body's tissues. Heart disease is a leading cause of death worldwide, and many patients require heart transplants to survive. However, there is a shortage of donor hearts.

### SUMMARY OF THE DISCLOSURE

In an aspect, a method of manufacturing autologous cardiac lineage cells is described. The method includes receiving a patient-specific sample from a subject, producing a plurality of fibroblast cells as a function of the patient-specific sample, wherein producing the plurality of fibroblast cells includes transferring the patient-specific sample to a first growth media, generating a plurality of induced pluripotent stem cells (iPSCs) as a function of the plurality of fibroblast cells, wherein generating the plurality of iPSCs includes identifying a plurality of confluent iPSCs from the plurality of iPSCs, and differentiating the plurality of iPSCs into a plurality of cardiac lineage cells, wherein differentiating the plurality of iPSCs into a plurality of cardiac lineage cells includes performing a two-dimensional (2D) expansion process on the plurality of confluent iPSCs and performing a three-dimensional (3D) expansion process on the plurality of 2D-expended confluent iPSCs.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram illustrating an exemplary method of manufacturing autologous cardiac lineage cells;

FIG. 2A-C are diagrams illustrating an exemplary method of manufacturing autologous cardiac lineage cells;

FIG. 3 is a diagram illustrating an exemplary method of collecting, expanding, and freezing fibroblast cells;

FIG. 4 is a diagram illustrating an exemplary method of reprogramming cells from a starting point of frozen fibroblast cells;

FIG. 5 is a diagram illustrating viral delivery of a polynucleotide to a cell;

FIG. 6 is a diagram illustrating an exemplary method of expanding iPSCs for differentiation;

FIG. 7 is a diagram illustrating an exemplary method of differentiating iPSCs into the plurality of cardiac lineage cells;

FIG. 8 is a diagram illustrating an exemplary method of manufacturing autologous cardiac lineage cells;

FIG. 9 is a diagram illustrating an exemplary system for automating a method of manufacturing autologous cardiac lineage cells; and

FIG. 10 is a diagram depicting elements of an exemplary computer system.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to a method of manufacturing autologous cardiac lineage cells. In an embodiment, the method includes generating induced pluripotent stem cells (iPSCs). Aspects of the present disclosure can be used to differentiate iPSCs into specialized cells such as, without limitation, cardiac lineage cells. Aspects of the present disclosure can also be used for transplantation of cardiac lineage cells. This is so, at least in part, because iPSCs are generated as a function of fibroblast cells produced from a patient-specific sample received via a skin biopsy performed on a subject. Such differentiated cells may be autologous with respect to the subject. Exemplary embodiments illustrating aspects of the present disclosure are described below in the context of several specific examples.

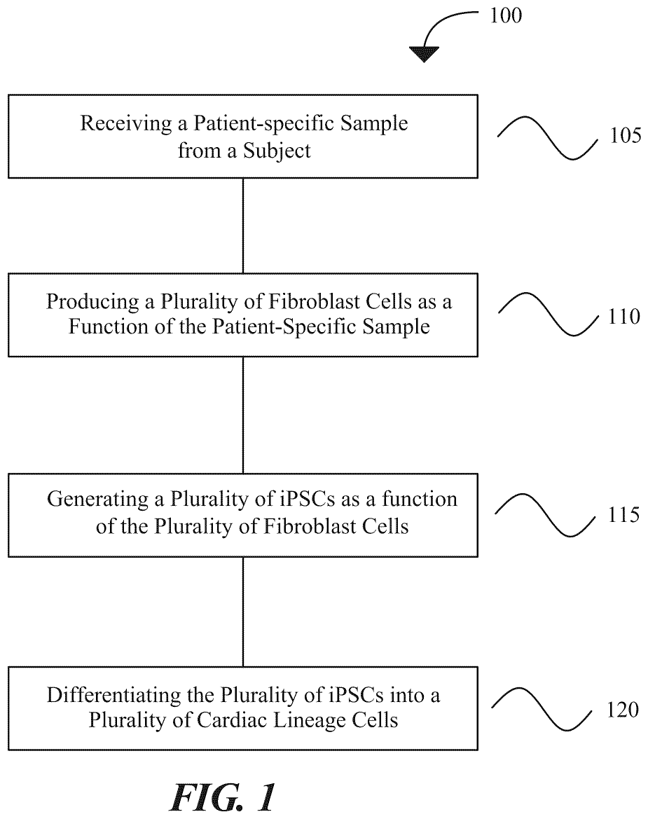

Now referring to FIG. 1, an exemplary method 100 of manufacturing autologous cardiac lineage cells is illustrated. As used in this disclosure, “cardiac lineage cells” are specialized cells that originate from the mesoderm and are primarily responsible for the formation, structure, and function of the heart. In some cases, cardiac lineage cells are vital for the development and maintenance of the cardiovascular system. In an embodiment, cardiac lineage cells may include cardiomyocytes, wherein the cardiomyocytes are the primary working cells of the heart, responsible for generating the contractile force needed for the heart to pump blood throughout the body. Cardiomyocytes may generate a contractile force needed for the heart to pump blood throughout the body. In another embodiment, cardiac lineage cells may include endothelial cells, wherein the endothelial cells are cells form the inner lining of blood vessels and the heart. Endothelial cells may maintain blood vessel integrity, regulate blood flow, and preventing clot formation. In a further embodiment, cardiac lineage cells may include smooth muscle cells, wherein the smooth muscle cells are cells surround blood vessels (e.g., in the tunica media layer of blood vessels). Smooth muscle cells may provide structural support, regulating blood flow, and pressure by contracting and relaxing.

With continued reference to FIG. 1, “Autologous,” for the purpose of this disclosure, refers to a biological material or treatment derived from the same subject who will receive it. In a non-limiting example, cells, tissues, or other biological components may be autologous when they are taken from a subject and then reintroduced into the same subject, either after being processed, modified, or expanded in vitro using methods/steps described anywhere in this disclosure. In some embodiments, autologous procedure may minimize the risk of immune rejection or complications related to donor incompatibility since the material is derived from the patient's own body. “Autologous cardiac lineage cells,” as described herein, are cardiac lineage cells, such as, without limitation, cardiomyocytes, endothelial cells, smooth muscle cells, and the like that are obtained from the same subject or otherwise generated using samples taken from the same subject who will receive them as part of an (regenerative) treatment or therapy via disclosed methods/steps described below. In some embodiments, treatment or therapy using autologous cardiac lineage cell may hold promise for treating various cardiovascular diseases and promoting heart repair and regeneration.

With continued reference to FIG. 1, method 100 includes a step 105 of receiving a patient-specific sample from a subject. As used in this disclosure, a “patient-specific sample” is a biological specimen obtained from a subject. A “subject,” as described herein, refers to an individual or patient (human or animal). Subject may be characterized by various factors such as, without limitation, age, sex, genetic background, medical history, presence or absence of specific health conditions, and the like. In a non-limiting example, subject be used to denote a human patient. In another non-limiting example, subject may include animal subjects such as, without limitation, mice, rats, monkeys, chimpanzees, and the like. In some embodiments, patient-specific sample may serve as a source of cells or genetic material used in manufacturing of autologous cardiac lineage cells as described in further detail below. In some cases, patient-specific samples may ensure compatibility between produced cells and subject's immune system, reducing the risk of rejection and adverse immune responses.

With continued reference to FIG. 1, in some embodiments, receiving the patient-specific sample may include receiving the patient-specific sample via a skin biopsy. In a non-limiting example, patient-specific sample may include a sample of skin tissue of subject. Such sample of skin tissue may include a plurality of fibroblast cells as described in further detail below. As used in this disclosure, a “skin biopsy” is a medical procedure in which a small sample of skin tissue is removed for further processing steps as described below. Skin biopsy may be manually performed by a professional such as a lab operator. In some cases, skin biopsy may include, without limitation, shave biopsy, punch biopsy, excisional biopsy, and the like.

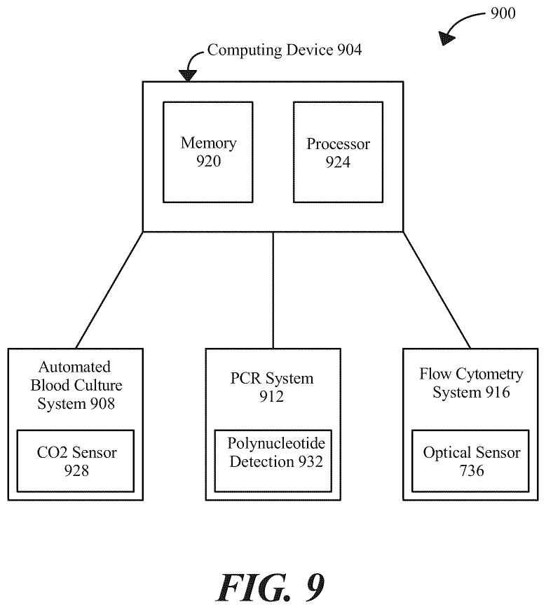

With continued reference to FIG. 1, method 100 includes a step 110 of producing a plurality of fibroblast cells as a function of the patient-specific sample. As used in this disclosure, “fibroblast cells” are a type of connective tissue cell found in various organs and tissues throughout the body. In some embodiments, fibroblast cells may maintain the structural integrity of tissues by producing extracellular protein matrix (ECM) components, such as, without limitation, collagen, elastin, glycosaminoglycans, and the like. In a non-limiting example, fibroblasts may include spindle-shaped cells with a large, flat nucleus and an elongated cytoplasm containing various organelles, such as, without limitation, endoplasmic reticulum, Golgi apparatus, and the like, which are involved in protein syntheses and secretion. These cells may be embedded within the ECM, where they may interact with other cells and matrix components to form a complex network that provides mechanical support, elasticity, and resilience to the tissues. Producing plurality of fibroblast cells may include cutting patient-specific sample with scalpels into a plurality of smaller pieces of patient-specific sample. Such procedure may be complete manually by a professional such as a lab operator, or automatically (i.e., mechanically) by a device/system for automating the method manufacturing autologous cardiac lineage cells as described below in reference to FIG. 9.

With continued reference to FIG. 1, “Extracellular protein matrix,” for the purpose of this disclosure, is a complex network of proteins, glycoproteins, and polysaccharides that provides structural support, biochemical and mechanical cues, and helps maintain tissue integrity in multicellular organisms. In some cases, ECM may include fibrous proteins, wherein the fibrous proteins may provide structural support and tensile strength to the ECM. In some cases, ECM may include proteoglycans, wherein the proteoglycans may include a core protein (i.e., glycosaminoglycans) linked to long chains of polysaccharides, contributes to ECM's viscoelastic properties, hydration, and resistance to compression. Glycosaminoglycans (GAGs) such as, without limitation, hyaluronic acid, chondroitin sulfate, heparan sulfate, and the like, may attract and bind water molecules, providing hydration and resistance to compression in the ECM. In some cases, ECM may include glycoproteins (e.g., laminin, entactin, tenascin, and the like), wherein the glycoproteins may be attached carbohydrate chains that play a role in cell signaling, adhesion, and migration.

With continued reference to FIG. 1, step 110 of producing the plurality of fibroblast cells includes transferring patient-specific sample to a first growth media. Transferring patient-specific sample may include transferring each pieces of plurality of pieces of patient-specific sample into a well. A “well,” as described herein, is an individual compartment within a microplate designed for holding samples (i.e., patient-specific sample) in a laboratory setting. In a non-limiting example, patient-specific sample may be cut into 6 pieces and placed into 6 wells, wherein each well may contain first growth media. As used in this disclosure, a “growth media” is a mixtures of nutrients and other components designed to support the growth, proliferation, and maintenance of cells in vitro, or outside the organism. In a non-limiting example, growth media may include a culture media that provide an optimal environment for cell (i.e., patient-specific samples) survival and function. Growth media may include a liquid media, wherein the liquid media are aqueous solutions containing a variety of nutrients and other components. Cutting patient-specific sample may create a larger surface area, which may facilitate better contact between patient-specific samples and first growth media. In a non-limiting example, first growth media may include a biopsy collection media. Patient-specific sample may be placed in a biopsy collection media, wherein the biopsy collection media may include a mixture of MEM, Antibiotic-Antimycotic, and Gentamicin.

With continued reference to FIG. 1, step 110 of producing plurality of fibroblast cells may include expanding plurality of fibroblast cells. As used in this disclosure, “expanding” refers to increasing the number of cells being expand in vitro by promoting their growth and proliferation in a controlled environment, typically within a tissue culture vessel, such as, without limitation, a flask, dish, multiwell plate, and the like. In some embodiments, plurality of fibroblast cells may consume nutrients from first growth media during the expansion process, thereby leading to accumulation of waste products; therefore, first growth medium may be refreshed regularly to maintain optimal cell health and support continued expansion of plurality of fibroblast cells.

With continued reference to FIG. 1, in some embodiments, plurality of fibroblasts may undergo a first expansion step. Plurality of fibroblasts may be placed into a plurality of wells containing first growth media. First growth media may include a fibroblast media, wherein the fibroblast media may include one or more of CTS knockout DMEM, CTS Glutamax, and PLTGold. Such growth media may allow fibroblasts in patient-specific sample to multiply. A first expansion step may include one or more medium changes. First expansion step may include medium changes, for example, on days 6, 12, and 18 post initial processing of patient-specific sample (i.e., cutting patient-specific sample as described above). First expansion step may include transfer/passage of a plurality of fibroblasts into a vessel or division of a plurality of fibroblasts into multiple vessels once plurality of fibroblasts reaches a specific confluency or density (e.g., around 80˜90% confluency) to prevent overcrowding and maintain their exponential growth phase. In some cases, vessels may contain ECM as described above. For example, a plurality of fibroblasts may be transferred into a T25 flask on day 9 post initial processing step. For example, a plurality of fibroblasts may be divided into 4 T25 flasks on day 15 post initial processing step. In some embodiments, passaging of plurality of fibroblasts may include detaching plurality of fibroblasts from the culture surface (i.e., the surface of the vessel) via a cell dissociation reagents such as, without limitation, TypLE dissociation reagent and transferring a selected number of cells to a new vessel with fresh first growth media.

With continued reference to FIG. 1, plurality of fibroblasts may be frozen to maintain their viability, functionality, and genetic stability over time. In some cases, plurality of fibroblasts cells my be cryopreserved, or stored at extremely low temperatures; for instance, and without limitation, plurality of fibroblasts cells may be placed in liquid nitrogen at −196 C or in a −80 C freezer with a freezing medium. Plurality of fibroblasts may be frozen, for example, on day 21 post initial processing step. Plurality of fibroblasts may be frozen in freezing medium, such as Cryotor CS10.

With continued reference to FIG. 1, method 100 includes a step 115 of generating a plurality of induced pluripotent stem cells (iPSCs) as a function of the plurality of fibroblast cells. As used herein, “induced pluripotent stem cells” (iPSCs) are a type of pluripotent stem cells, where a “pluripotent stem cell” is a type of undifferentiated cells that can be generated from adult somatic cells, such as skin or blood cells, through reprogramming, and have ability to develop into any of the germ primary germ layers in an organism: ectoderm, mesoderm, and endoderm. These germ layers may give rise to all the different cell types and tissues in the body. In some embodiments, iPSCs may include properties similar to those of embryonic stem cells (ESC) and encompasses undifferentiated cells artificially derived by reprogramming differentiated, non-pluripotent cells. A “differentiated” cell, for the purpose of this disclosure, is a cell that takes on a more committed (“differentiated”) position within a given cell lineage.

With continued reference to FIG. 1, as used herein, the term “reprogramming” means a process that alters or reverses the differentiation status of a somatic cell that is either partially or terminally differentiated. Reprogramming of a somatic cell may be a partial or complete reversion of the differentiation status of the somatic cell. In some embodiments, reprogramming is complete when a somatic cell is reprogrammed into an induced pluripotent stem cell. However, reprogramming may be partial, such as reversion into any less differentiated state. For example, reverting a terminally differentiated cell into a cell of a less differentiated state, such as a multipotent cell. As used herein, the term “somatic cell” refers to any cell other than pluripotent stem cells or germ cells. In some embodiments, a somatic cell is a fibroblast.

Still referring to FIG. 1, a plurality of fibroblasts may be thawed. As used in this disclosure, “thawing” refers to a process of warming up cryopreserved cells to bring them back to a viable state for further processing steps of method 100. For example, and without limitation, a plurality of fibroblasts may be thawed, for example, on day 6 before infection.

Still referring to FIG. 1, a plurality of fibroblasts may undergo a second expansion step, such as by being placed into one or more wells containing a second growth medium. A second growth medium may include fibroblast media as described above; for instance, and without limitation, second growth medium may include one or more of CTS knockout DMEM, CTS Glutamax, and PLTGold. A second expansion step may allow fibroblasts and/or iPSC in a sample to multiply.

Still referring to FIG. 1, a plurality of fibroblasts may be infected with one or more viral vectors encoding one or more reprogramming factors. As used in this disclosure, a “viral vector” is a genetically engineered virus that has been modified to carry foreign genetic material, such as a specific gene or a small RNA molecule, into host cells. In an embodiment, viral vector may be capable of efficiently infecting target cells (i.e., plurality of fibroblasts) and delivering one or more desired genetic materials (i.e., reprogramming factors) to target cells without causing severe disease or harm to target cells/the organism. In a non-limiting example, reprogramming plurality of fibroblast cells may include delivering a polynucleotide encoding a reprogramming factor to the plurality of fibroblast cells via a viral vector. In some cases, viral vector may include an adenoviral vector, wherein the adenoviral vector are non-enveloped, double-stranded DNA viruses that can infect a wide range of host cells including dividing and non-dividing cells. In some cases, viral vector may include a retroviral vector, wherein the retroviral vector is enveloped, single-stranded RNA viruses that replicate through a DNA intermediate. In some cases, viral vector may include a lentiviral vector, wherein the lentiviral vector is a subclass of retroviruses with ability to infect both dividing and non-dividing cells. In a non-limiting example, viral vector may be from an RNA virus. RNA viruses do not require integration of viral DNA into a host genome. Plurality of fibroblasts may be infected with Sendai virus encoding one or more reprogramming factors. “Sendai virus,” as described herein, is a negative-sense, single-stranded RNA virus belonging to Paramyxoviridae family. Sendai virus (M-PIV1) is an enveloped virus and may be used as a viral vector for gene delivery and iPSC generation. In some embodiments, use of an RNA virus (rather than a DNA virus) may prevent the possibility of integration into an important host gene and allow viral genetic material to be more easily cleared.

Still referring to FIG. 1, a viral vector may encode one or more reprogramming factors. As used herein, the term “reprogramming factor” refers to a molecule, which when contacted with a cell, or produced in a cell from exogenous DNA or RNA (e.g., produced from transduced RNA), can, either alone or in combination with other molecules, cause reprogramming. In some cases, viral vector may encode a reprogramming factor selected from the list consisting of Oct3 protein, Oct4 protein, Myo-D-Oct4 protein, Sox1 protein, Sox2 protein, Sox3 protein, Sox15 protein, Klf1, protein, Klf2 protein, Klf3 protein, Klf4 protein, Klf5 protein, c-Myc protein, L-Myc protein, N-Myc protein, Nanog protein, Lin28A protein, Tert protein, Utf1 protein, Aicda protein, Glis1, Sa114, Esrrb, Tet1, Tet2, Zfp42, Prdm14, Nr5a2, Gata6, Sox7, Pax1, Gata4, Gata3, cEBPa, HNF4a, GMNN, SNAIL, Grb2, Trim71, and biologically active fragments, analogues, variants, and family members thereof. Reprogramming factors may be able to convert adult cells into iPSCs. In an embodiments, reprogramming factors may be introduced into plurality of fibroblasts via one or more viral vector. For example, and without limitation, a viral vector may encode one or more of hOct3/4, hSox2, hKlf4, hc-Myc, GMP sendai virus and/or hl-Myc. Exposure of a plurality of fibroblasts to a viral vector encoding one or more reprogramming factors may result in reprogramming of fibroblasts into iPSC.

Still referring to FIG. 1, second expansion step may include one or more medium changes. For example, a medium may be changed, for example, on day 4 before infection. For example, media may be changed on days 1, 2, 4, and 6 post infection. A plurality of fibroblasts or a plurality of iPSC may be replated during a second expansion step. For example, a plurality of fibroblasts may be replated on day 2 before infection. A plurality of fibroblasts or a plurality of iPSC may be transferred to a plate containing an extracellular protein matrix (such as laminin, collagen, fibronectin, etc.). For example, a plurality of fibroblasts or a plurality of iPSC may be transferred to a plate containing a laminin 521 matrix. A plurality of fibroblasts or a plurality of iPSC may be transferred to a plate containing laminin 521, for example, on day 7 post infection. Media changes and cell division may aid in clearing or diluting a portion of a viral vector. In some embodiments, media changes may be done until the viral vector has been cleared. A plurality of fibroblasts or a plurality of iPSC may adhere to an intracellular protein matrix such that the plurality of fibroblasts or the plurality of iPSC is retained on a plate through a medium change.

Still referring to FIG. 1, a medium in a vessel containing a plurality of fibroblasts or a plurality of iPSC may be changed to a reprogramming media. In a non-limiting example, reprogramming plurality of fibroblast cells may include transferring plurality of fibroblast cells to reprogramming media. As used herein, a reprogramming media is a medium that is conducive to iPSC expansion. In some embodiments, exposure of fibroblasts to reprogramming factors and culture in reprogramming media causes fibroblasts to be reprogrammed into iPSC and expand. In some cases, reprogramming media may include one or more of L-ascorbic acid-2-phosphate magnesium, sodium selenium, FGF2, insulin, NaHCO3, transferrin, TGFβ1, and NODAL. In some embodiments, reprogramming media may be based on a DMEM/F12 medium. Exemplary reprogramming media include mTeSR1, STEMPRO, Tesr E7 and Tesr E8. In some embodiments, reprogramming media may be configured to cause iPSC reprogrammed from fibroblasts via reprogramming factors to expand. In some embodiments, cell expansion includes cell growth and multiplication, and may be caused by, for example, and without limitation, culturing cells in a medium containing nutrients and a matrix. Second growth media in a vessel containing plurality of fibroblasts or plurality of iPSCs may be changed to reprogramming media on day 8 post infection.

With continued reference to FIG. 1, a plurality of iPSC may be characterized, and a plurality of iPSCs may be selected and subjected to 2D expansion. For example, 12 iPSCs clones may be selected and subjected to a two-dimensional (2D) expansion process. iPSCs colonies may be selected and transferred, for example, on day 21-30 post infection. As used in this disclosure, a “2D expansion process” is an in-vitro cultivation and propagation of cells (i.e., iPSCs) on a 2D surface using cell culture plates or flasks. In a non-limiting example, 2D expansion of plurality of iPSCs may include facilitating the growth of iPSCs along an XY axis. 2D expansion process may include transferring iPSC into one or more vessels containing a 2D culture media. A 2D culture media may include an Mtesr1 medium. Vessel containing 2D culture medium may include ECM (such as laminin, collagen, fibronectin, etc.) as described above; for instance, and without limitation, the surface of vessel containing a 2D culture media may be coated with a laminin 521 matrix.

Still referring to FIG. 1, 2D expansion process may include passaging plurality of iPSCs into one or more vessels and/or one or more media changes. For example, on day 35 post infection, 6 clones may be transferred into a 6 well plate. For example, on day 40 post infection, 6 clones may be transferred into a 60 mm plate. For example, on day 40-70 post infection, 6 clones may be cycled in a 60 mm plate. For example, from day 75-85 post infection, 3 clones may be expanded from 1 to 2 to 4 to 12 plates. In some embodiments, 2D expansion process may improve confluence, morphology, cleanliness and clear viral vector. Plurality of iPSC may be separated, such as for passage to a different vessel, using a cutting apparatus.

With continued reference to FIG. 1, plurality of iPSCs may be tested post-2D expansion process. For example, a plurality of iPSC may undergo one or more of sterility, mycoplasma, karyotype, DNA fingerprinting, residual virus, pluripotency marker, etoposide sensitivity, and thaw grade tests. In an embodiment, iPSC may undergo a sterility test. A sterility test may reveal whether microorganisms are present in an iPSC sample. A sterility test may measure CO2 levels in a sample in order to determine whether microorganisms are present. For example, a CO2 sensor may be used to detect CO2 levels periodically, with an increasing rate of change in CO2 levels indicating the presence of microorganisms. In some embodiments, a sterility test may be done using an automated blood culture system. Automated blood culture may be done using, for example, a VersaTREK automated microbial detection system. In some embodiments, a sterility test may have a negative result. In some embodiments, one or more processes of a sterility test is automated. In some embodiments, incubation and/or CO2 detection is automated, such as via a fluorescent CO2 sensor.

Still referring to FIG. 1, in an embodiment, iPSCs may undergo a mycoplasma test. A mycoplasma test may reveal whether mycoplasma are present in an iPSC sample. Mycoplasma are very small self-replicating bacteria that may cause changes in cell membranes, nucleic acid and amino acid metabolism, rates of cell growth, and may cause chromosomal defects. Mycoplasma may be detected via direct culture, DNA staining with fluorescent dye, hybridization of nucleic acid, biochemical tests, and PCR. In some embodiments, a mycoplasma test may be done using PCR, for example, using primers associated with a mycoplasma gene. In some embodiments, a mycoplasma test has a negative result. In some embodiments, one or more processes of a mycoplasma test is automated. For example, various aspects of PCR may be automated, such as sample lysis, polynucleotide amplification, and polynucleotide detection.

Still referring to FIG. 1, in an embodiment, iPSCs may undergo a karyotype test. A karyotype test may measure genomic integrity of an iPSC sample. A karyotype test may be done by G-banding. In some embodiments, G-banding includes staining chromosomes, and visually examining stained metaphase chromosomes for abnormalities such as varying chromosome count or large deletions, insertions, or translocations. In some embodiments, a karyotype test may have a normal result.

Still referring to FIG. 1, in an embodiment, iPSCs may undergo a DNA fingerprinting test. A DNA fingerprinting test may measure a genotype of an iPSC sample and compare it to a genotype of a parent fibroblast sample. A DNA fingerprinting test may be done using a short tandem repeat (STR) system. In some embodiments, an STR system measures STRs, short DNA sequences repeated in a genome, and determines whether the length of fibroblast STRs match iPSC STRs. In some embodiments, a DNA fingerprinting test may indicate that there is a match with the plurality of fibroblasts the plurality of iPSC was derived from. In some embodiments, PCR may be used to determine STR length. In some embodiments, one or more processes of a DNA fingerprinting test is automated. For example, various aspects of PCR may be automated, such as sample lysis, polynucleotide amplification, and polynucleotide detection.

Still referring to FIG. 1, in an embodiment, iPSCs may undergo a residual virus test. A residual virus test may measure an amount of viral polynucleotide in an iPSC sample. A residual virus test may measure an amount of viral polynucleotide from a viral vector encoding one or more reprogramming factors. For example, and without limitation, in the case of an RNA viral vector from Sendai virus as described above. Residual virus test may measure an amount of Sendaiviral RNA in an iPSC sample. In some embodiments, PCR may be used to measure viral DNA (for example, using viral primers). In some embodiments, reverse transcriptase PCR may be used to measure viral RNA (for example, using viral primers). In some embodiments, virus is not detected. In some embodiments, virus is not detected after 40 cycles. In some embodiments, one or more processes of a residual virus test is automated. For example, various aspects of PCR may be automated, such as sample lysis, polynucleotide amplification, and polynucleotide detection.

Still referring to FIG. 1, in an embodiment, iPSCs may undergo a pluripotency marker test. A pluripotency marker test may measure the amount of cells in a sample that have pluripotent cell (or iPSCs) markers. In some cases, pluripotency marker test measures the percent of cells in a sample that express TRA-1-60 and TRA-1-81. In some embodiments, TRA-1-60 and TRA-1-81 are markers for human pluripotent stem cells, such as iPSCs. In some embodiments, a pluripotency marker test may be done via flow cytometry (such as using fluorescent antibodies targeting TRA-1-60 and TRA-1-81). In some embodiments, >70% of cells in a sample may express pluripotency markers. In some embodiments, one or more processes of a pluripotency marker test is automated. For example, various aspects of flow cytometry may be automated, such as capturing images of cells, and analyzing those images (such as for fluorescence).

Still referring to FIG. 1, in an embodiment, iPSCs may undergo an etoposide sensitivity test. An etoposide sensitivity test may measure cell viability using flow cytometry after exposure of cells to etoposide. In some embodiments, iPSCs are more susceptible to DNA damaging agents than differentiated cells. In some embodiments, iPSCs have lower cell viability after exposure to etoposide. In some embodiments, etoposide is a DNA damaging agent. In some embodiments, an alternative DNA damaging agent, such as topoisomerase II, may be used. In some embodiments, an etoposide sensitivity test may have a EC50<300 nM result. In some embodiments, an etoposide sensitivity test may be used to determine product quality. In some embodiments, one or more processes of a pluripotency marker test is automated. For example, various aspects of flow cytometry may be automated, such as capturing images of cells, and analyzing those images (such as for cell viability).

Still referring to FIG. 1, in an embodiment, iPSCs may undergo a thaw grade test. A thaw grade test may include a relative determination of iPSCs quality based on manual observational assessment. In some embodiments, a thaw grade test may be done by a computer system, such as an artificial intelligence system.

Still referring to FIG. 1, generating the plurality of iPSCs may include identifying a plurality of confluent iPSCs from the plurality of iPSCs. As used in this disclosure, “confluent” refers to a state in which iPSCs have proliferated and covered the entire surface of the culture dish or plate with individual cells touching each other to form a continuous monolayer. In some embodiments, a criteria may include selecting cloned cells based on criteria such as cells that are confluent, have a certain nucleus to cytoplasm ratio, no genomic instability, no DNA damage, “solid edges,” a “homogenous appearance,” and the like. In an embodiment, iPSC may be graded using a criteria, wherein confluent iPSC may be selected as function of the grade. In some instances, this may be done by observational reports done by a human, in other instances this may be done using computer software technology. In some embodiments, plurality of iPSCs may be selected based on a grade. In some embodiments, plurality of iPSCs may be selected based on the results of one or more tests selected from sterility, mycoplasma, karyotype, DNA fingerprinting, residual virus, pluripotency marker, etoposide sensitivity, and thaw grade tests as described above. In some embodiments, a plurality of confluent iPSC may be selected for further processing steps of method 100.

With continued reference to FIG. 1, plurality of iPSCs and/or selected confluent iPSCs may be frozen. For example, and without limitation, plurality of confluent iPSCs may be frozen on day 90 post-infection. In some cases, plurality of iPSCs and/or confluent iPSCs may be frozen in freezing media as described above, such as Cryotor CS10.

With continued reference to FIG. 1, method 100 includes a step 120 of differentiating plurality of iPSCs into a plurality of cardiac lineage cells. As used in this disclosure, “differentiating” refers to a process by which unspecialized, multipotent, or pluripotent cells (i.e., iPSCs) undergo a series of molecular and morphological changes to acquire specialized functions, characteristics, and phenotypes that are specific to a particular cell lineage or tissue type. In an embodiment, differentiating plurality of iPSCs into plurality of cardiac lineage cells may be governed by a coordinated action of various signaling pathways. As used in this disclosure, “signaling pathways” are a series of molecular events and interactions that occur within a cell to transmit information from the cell surface to the nucleus or other cellular components. In a non-limiting example, each iPSC of plurality of iPSCs may include receptors located on the cell surface or within the cell that recognize and bind to specific signaling molecules such as ligands, thereby initiating a signaling cascade. Intracellular signaling molecules (e.g., enzymes, adapter proteins, second messengers, and the like) may be activated once receptor binds to ligand. Intracellular signaling molecules may be responsible for propagating the signal within the each iPSC. Signaling path way may further include effector proteins, wherein the effector proteins may carry out the intended cellular response such as, without limitation, activating gene transcription, inducing cell division, triggering apoptosis, and/or the like. In some embodiments, signaling pathways may be highly regulated and interconnected, allowing plurality of iPSCs to respond to their environment, communicate with each other, and/or maintain homeostasis.

With continued reference to FIG. 1, in some embodiments, plurality of iPSCs may be differentiated into a cell type selected from the list consisting of cardiac lineage cells, lung cells, liver cells, stomach cells, kidney cells, muscle cells, pancreas cells, skin cells, cartilage cells, and embryonic cells.

With continued reference to FIG. 1, plurality of iPSCs may be thawed. In some embodiments, plurality of confluent iPSCs identified and selected from plurality of iPSCs may be thawed. Differentiating plurality of iPSC into cardiac lineage cells include performing 2D expansion process as on plurality of confluent iPSCs. In a non-limiting example, plurality of confluent iPSC may be thawed and plated on a single plate on day 0, expand and split (via dissociation reagent such as, without limitation, Accutase) into 2 plates on day 5, expand and split into 4 plates on day 10, and split into 12 plates on day 15. Plates may contain an adhesion protein such as laminin 521. Plates may include 2D culture media as described above, which may include Mtesr1.

With continued reference to FIG. 1, differentiating iPSCs into cardiac lineage cells includes performing a three-dimensional (3D) expansion process on plurality of 2D-expanded confluent iPSCs. As used in this disclosure, a “3D expansion process” is an in-vitro cultivation and propagation of cells (i.e., iPSCs) within a 3D extracellular matrix (ECM) or scaffold that provides a biomimetic environment, facilitating cell-cell and cell-matrix interactions, and closely resembling the native tissue architecture. In a non-limiting example, performing 3D expansion process on plurality of confluent iPSCs may include facilitating the growth of the confluent iPSCs along a XYZ axis (3D culture system). Such 3D culture system may provide a more physiologically relevant environment that better mimics the structure and function of tissues in vivo. After 2D expansion process as described above, plurality of 2D-expanded confluent iPSCs may be transferred between various size of vessels, depending on the size of 3D expanding confluent iPSCs; for instance, and without limitation, plurality of 2D-expanded confluent iPSCs may be passaged into to a 50 mL vessel on day 20 post thawing for 3D expansion, passaged into a 100 mL vessel on day 23 post thawing, passaged into a 200 mL vessel on day 26 post thawing, passaged into a 400 mL vessel on day 29 post thawing, and passaged into a 800 mL vessel on day 32 post thawing. Plurality of 2D-expanded confluent iPSCs may be in a 3D culture media during 3D expansion process. In a non-limiting example, 3D culture media may include one or more of Mtesr, DMEM/F12+Glutamax, StemProhESC supplement, BSA 25%, bFGF, and 2-ME. Each passage of plurality of 2D-expanded confluent iPSCs may include three-day cycle of media change. For example, and without limitation, three-day cycle of media change (i.e., replacement of 3D culture media) may include 50% media change on day 1, 80% media change on day 2, and passage of plurality of 2D-expanded confluent iPSCs on day 3. A Pre-differentiation (pre-diff) passage of plurality of 3D-expanded confluent iPSCs to a 500 mL vessel may be performed on day 33 after the last passage on day 32 post thawing.

With continued reference to FIG. 1, differentiating the plurality of iPSCs into the plurality of cardiac lineage cells may include transferring plurality of confluent iPSCs to a differentiation media. In an embodiment, transferring plurality of confluent iPSCs may occur after last passage or pre-diff passage, for example, on day 33; for instance, and without limitation, plurality of confluent iPSCs may include plurality of 3D-expanded confluent iPSCs as described above. Differentiation media may include specific combination of growth factors, signaling molecules, and other supplements that promote the directed differentiation of iPSCs into specific cell types or lineages such as, without limitation, cardiac lineage cells. In a non-limiting example, differentiation media may include a basal media containing a Roswell Park Memorial Institute medium (RPMI) with B27+/B27− supplement (i.e., an optimized serum-free supplement used to support the low- or high-density growth and short- or long-term viability of embryonic, post-natal, and adult hippocampal and other CNS neurons), as produced by Thermo Fischer Scientific, Inc. As used in this disclosure, a “basal media” is a nutrient-rich solution that provide the basic components necessary to support the growth and maintenance of cells in culture. In some cases, basal media may be supplemented with additional components such as growth factors, hormones, serum, insulin and/or the like to support the differentiation of plurality of iPSCs. In a non-limiting example, transferring the plurality of confluent iPSCs to differentiation media may include adding a supplementary media into the differentiation media, wherein the supplementary media is configured to modulate a Wnt signaling pathway. As used in this disclosure, a “Wnt signaling pathway” is a conserved cell signaling system in biological processes. In some cases, biological processes may include, without limitation, embryonic development, cell proliferation, differentiation, migration, tissue homeostasis, tissue regeneration, and the like.

Still referring to FIG. 1, in some cases, supplementary media may include a first inhibitor containing CHIR. As used in this disclosure, an “inhibitor” is a molecule that binds to a specific target, such as an enzyme or a receptor, and reduces or prevents its activity (i.e., signaling pathway) In a non-limiting example, inhibitor may be naturally occurring or synthetic molecules. Inhibitor may be used to modulate cellular processes by selectively blocking or dampening the activity of specific proteins or signaling pathways. “CHIR (CHIR99021),” for the purpose of this closure, is a small molecule inhibitor of glycogen synthase kinase 3 (GSK-3), wherein the GSK-3 is a serine/threonine protein kinase involved in various cellular processes, such as cell signaling, proliferation, differentiation, apoptosis, and the like. CHIR99021 may be used to promote mesoderm formation and cardiomyocyte differentiation. In a non-limiting example, CHIR99021 may act by selectively inhibiting both the alpha and beta isoforms of GSK-3, thereby leading to the activation of the Wnt signaling pathway. CHIR99021 may be added to basal media at day 0 after pre-diff passage.

Still referring to FIG. 1, in some cases, supplementary media may include a second inhibitor containing IWP 4. As used in this disclosure, an “IWP 4 (inhibitor of Wnt Production-4)” is a small molecule inhibitor that selectively blocks the secretion and activity of Wnt proteins. In a non-limiting example, IWP-4 may inhibit the membrane-bound O-acyltransferase porcupine, an enzyme essential for the maturation and secretion of Wnt ligands, thereby preventing the secretion of Wnt proteins and modulate Wnt signaling pathway. Additionally, or alternatively, inhibitors may be used in combination. In a non-limiting example, IWP-4 may be sued in combination with other growth factors and signaling molecules to direct the differentiation of plurality of iPSCs into cardiac lineage cells; for instance, and without limitation, IWP-4 may be used to inhibit Wnt signaling at specific stages of the differentiation process, working in conjunction with other factor such as, without limitation, activin A, bone morphogenetic protein 4 (BMP4), CHIR99021, and the like to guide iPSCs toward a cardiac lineage. IWP 4 may be added to basal media at day 3 after pre-diff passage.

With continued reference to FIG. 1, differentiating plurality of iPSCs into plurality of cardiac lineage cells may further include washing the plurality of cardiac lineage cells with Dulbecco's phosphate-buffered saline (DPBS). Plurality of cardiac lineage cells may be harvested during or after washing the plurality of cardiac lineage cells. In an embodiment, plurality of cardiac lineage cells may be washed with DPBS to remove residual serum or media (i.e., differentiation media) components. In such embodiment, DPBS may prevent the accumulation of toxic byproducts. DPBS may contain essential ions such as, without limitation, calcium, magnesium, potassium, sodium, and the like. In an embodiment, plurality of cardiac lineage cells may be washed with DPBS to maintain plurality of cardiac lineage cells viability for instance, and without limitation, balanced salt solution contained in DPBS may mimics an ionic composition of extracellular fluid and provide the optimal environment for cell survival and function.

With continued reference to FIG. 1, differentiating plurality of iPSCs into plurality of cardiac lineage cells may include separating plurality of cardiac lineage cells into a plurality of discrete cellular objects. As used in this disclosure, a “discrete cellular object” is an individual cell or a group of cells (i.e., clump) thereof that are separated from other such objects and/or the tissue mass (e.g., a larger population of cells). In some cases, plurality of cardiac lineage cells may be separated into plurality of discreate cellular objects through a dissociation process using a dissociation media. As used in this disclosure, a “dissociation process” refers to a separation of discrete cellular object from discrete cellular object aggregates. In an embodiment, dissociation process may create a single-cell suspension from discrete cellular object aggregate such as, without limitation, plurality of cardia lineage cells being dissociated. As used in this disclosure, a “dissociation media” is a specialized solutions containing enzymes, chelating agents, and/or other components that facilitate the dissociation of discrete cellular objects from discrete cellular object aggregates. In a non-limiting example, dissociation media may include Liberase, DNase, sodium chloride, Uberase, Tryple, and the like that may be used to dissociate a population of cardiac lineage cells into a plurality of chumps or aggregates of cardiac lineage cells, rather than single cells. In some cases, plurality of discrete cellular objects may be separated from other discrete cellular objects (e.g., other cells or other clumps of cells). In other cases, dissociation process may include mechanical dissociation. Cell-cell and cell-matrix interactions of plurality of cardiac lineage cells may be physically disrupted using mechanical forces; for instance, and without limitation, via manual pipetting and the like.

With continued reference to FIG. 1, method 100 may further include a step of subjecting the plurality of cardiac lineage cells to a differentiation completion test and harvesting the plurality of cardiac lineage cells. As used in this disclosure, a “differentiation completion test” is a test used to finalize the completion (i.e., determine a degree) of differentiation of cells such as iPSCs described above. In a non-limiting example, differentiation completion test may include one or more tests selected from the list consisting of sterility, mycoplasma, karyotype, DNA fingerprinting, residual virus, pluripotency maker, etoposide sensitivity, and thaw grade tests.

With continued reference to FIG. 1, plurality of cardiac lineage cells may be frozen to maintain their viability, functionality, and genetic stability over time. Frozen cardiac lineage cells may also be used as reserve vials. Plurality of fibroblasts may be frozen, for example, on day 20 post adding first supplementary media to differentiation media. Plurality of cardia cardiac cells may be frozen in freezing medium, such as Cryotor CS10.

Still referring to FIG. 1, an iPSC generated by a method described herein may have a variety of applications and therapeutic uses. In some embodiments, a method disclosed herein may be used to reprogram cells suitable for therapeutic applications, including autologous transplantation into subjects.

Still referring to FIG. 1, the methods disclosed herein can be used to generate iPSC that can be further modulated to form any type of somatic cells by culturing the iPSC under cell-type specific conditions. Cell-type or cell lineage specific conditions may include contacting the iPSC with cell and cell lineage differentiation factors. Specifically, iPSC can be differentiated toward a neuronal lineage by exposing them to one or more factors that include, but are not limited to, N2 and B27 supplements, Noggin, SB431542, DMEM/F12 medium, laminin, cyclic adenosine monophosphate (cAMP), ascorbic acid, brain-derived neurotrophic factor (BDNF), glial cell-derived neurotrophic factor (GDNF), insulin-like growth factor I (IGF-I), fibroblast growth factor (FGF)-8, transforming growth factor (TGF) beta 3 (TGF-β3), or retinoic acid. iPSC can be differentiated toward an endodermal lineage (such as hepatocytes, pancreatic cells, intestinal epithelial, lung cells) by exposing them to specific differentiation factors and media, which include, but are not limited to, RPMI medium, SFD medium, N2/B27 medium, glutamine, monothioglycerol (MTG), CHIR 99021, activin A, ascorbic acid, bone morphogenetic protein (BMP)-4, vascular endothelial growth factor (VEGF), epidermal growth factor (EGF), basic FGF (bFGF), hepatocyte growth factor (HGF), dexamethasone, TGF-α, hydrocortisone, FGF-7, or Exendin-4. Cardiomyocyte lineage differentiation factors and media include, but are not limited to, StemPro medium, DMEM/F12 medium, BMP4, Activin A, bFGF, VEGF, Dickkopf-related protein 1 (DKK1), Transferrin, MTG, or ascorbic acid. For mesenchymal stem cell differentiation, iPSC can be exposed to fetal serum and differentiation factors which include, but are not limited to, bFGF, BMP-4, EGF, retinoic acid, or platelet derived growth factor (PDGF). iPSC-derived MSC can subsequently be differentiated toward (1) bone progenitors (osteocytes) through exposure to one or more factors such as ascorbic-acid-2-phosphate, β-glycerophosphate, M dexamethasone or BMP-2, (2) chondrogenic progenitor (chondrocytes) through exposure to one or more factors such as dexamethasone, ascorbic-acid-2-phosphate, proline, pyruvate, TGF-β3, or insulin/transferrin/selenious acid supplement (ITS) (3) adipogenic progenitors through exposure to one or more factors such as hydrocortisone, isobutylmethylxanthine or indomethacin; and (4) fibroblasts through exposure to connective tissue growth factor (CTGF). Fibroblasts can also be derived directly from iPSC via exposure to one or more factors such as TGF-β2, ascorbic acid, connective tissue growth factor (CTGF), ITS reagents, or fetal serum. Keratinocyte lineage differentiation factors include, but are not limited to, BMP4, retinoic acid, ascorbic acid, insulin, hydrocortisone, bovine pituitary extract, IGF-1 or EGF.

Still referring to FIG. 1, a method disclosed herein may also be useful to reprogram or dedifferentiate cells prior to re-differentiation of cells and organ formation. In some embodiments, the methods include generating organs by exposing pluripotent cells generated by the methods described herein to differentiating factors and combining one or more of the differentiated cells and cell types under conditions sufficient to encourage organ formation. For example, iPSCs generated using the methods described herein can be differentiated to cells that can be used to make skin as well as other organs such as liver, bones, and cartilage. Such methods include combining one or more of the lineages and/or cell types that form an organ under conditions sufficient to encourage organ formation. Specifically, conditions sufficient to form skin may include but are not limited to co-culture or in vivo co-grafting of iPSC-derived keratinocytes and fibroblasts. For ex vivo generated skin equivalents, fibroblasts are grown on extracellular protein matrix (such as collagen, laminin, fibronectin, etc.) to form a dermis-like structure followed by overlaying with keratinocytes to produce epidermis. For an in vivo generation of human skin equivalents/grafting, a silicone grafting chamber can be surgically inserted onto the muscle fascia of recipient severe combined immunodeficiency (SCID) mice. A cell slurry consisting of keratinocytes and fibroblasts derived from human iPSCs is introduced into this chamber. The cells and factors necessary to generate human skin equivalents ex vivo and in vivo include, but are not limited to, iPSC derived keratinocytes, fibroblasts, melanocytes and derma papilla cells, EGF, insulin, fetal serum, ascorbic acid, hydrocortisone, bovine pituitary extract, IGF-1, or DMEM medium. Bones can be grown ex vivo by culturing iPSC-derived osteocytes in the presence of ascorbic-acid-2-phosphate, β-glycerophosphate and fetal serum. Cartilage can be generated by culturing iPSC-derived chondrocytes as micromasses in the presence of ITS, dexamethasone, ascorbic-acid-2-phosphate, proline, pyruvate and TGF-β3. Liver can be generated via the formation of liver buds. Conditions sufficient to form liver buds may include, but are not limited to, the combination of mesenchymal stem cells with hepatic progenitors (both can be derived from iPSC as described above) in the presence of endothelial growth medium and/or hepatocyte culture medium supplemented with dexamethasone, oncostatin, HGF, and matrigel.

Still referring to FIG. 1, in some embodiments, a method for treating or preventing one or more symptoms of a disease or disorder in a subject may include reprogramming cells to pluripotency in vitro, differentiating the cells to one or more appropriate cell types, and administering a therapeutically effective amount of differentiated cells to a subject in need thereof. A method may include obtaining one or more somatic cells from a subject and reprogramming the cells into iPSCs or dedifferentiated cells. Cells may be cultured under conditions that allow for the cells to differentiate into a desired cell type suitable for treating or preventing a condition. Differentiated cells may be introduced into a subject to treat or prevent a condition. For example, a method may include obtaining one or more somatic cells from a subject, reprogramming the somatic cells into iPSCs, differentiating iPSCs into a desired cell type such as, without limitation, cardiac lineage cells, and administering a therapeutically effective amount of differentiated cells to the subject.

Still referring to FIG. 1, in some embodiments, the methods disclosed herein may yield reprogrammed cells with normal karyotypes or with karyotypes that are the same as the subject from whom they were derived. In some embodiments, uncorrected iPSCs from subjects may be differentiated into cell types relevant to a genetic disorder for modeling a disease. In another embodiment, a particular mutation of interest may be introduced into normal healthy iPSCs, as another approach to modeling a disorder.

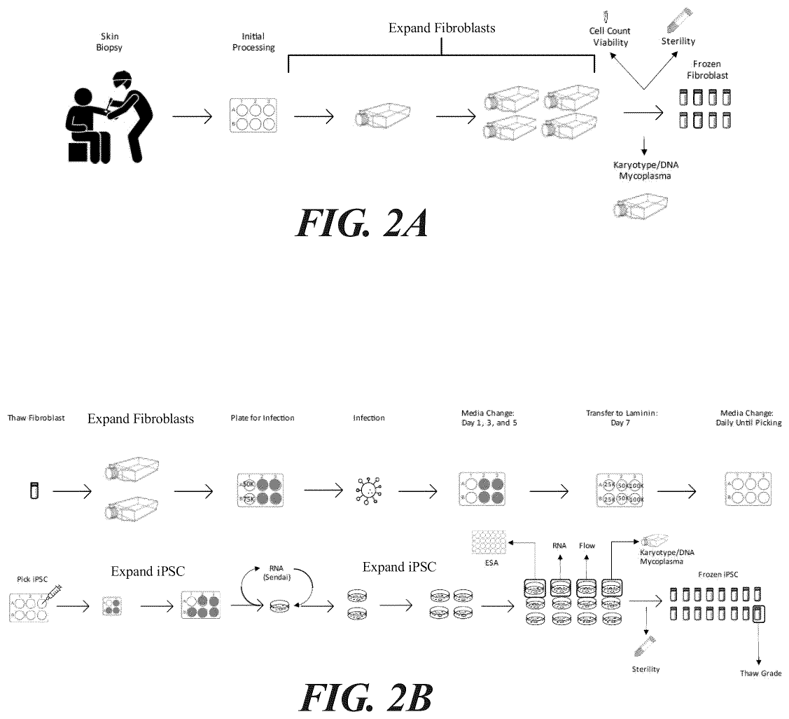

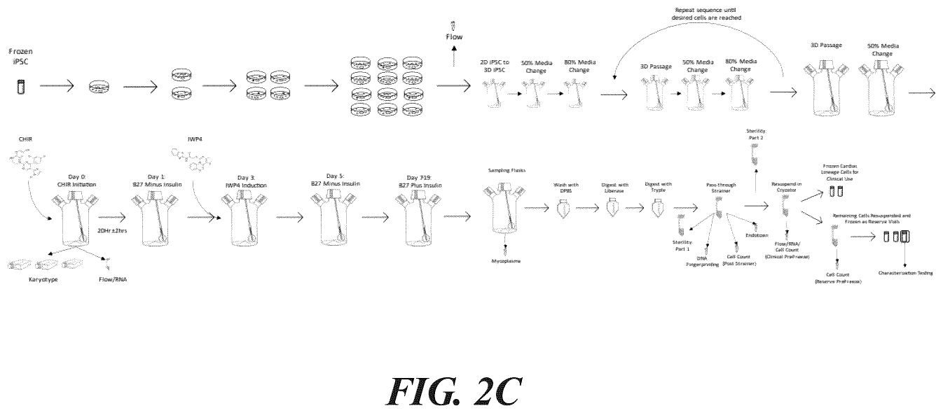

Now referring to FIG. 2A, in some embodiments, a method includes, in the following order, receiving fibroblasts from a skin biopsy, an initial processing step, an expansion step, and a testing step. In some embodiments, fibroblasts are then frozen.

Now referring to FIG. 2B, in some embodiments, a method includes, in the following order, thawing fibroblasts, expanding fibroblasts, plating fibroblasts for infection, infecting fibroblasts, media changes, transfer to a laminin plate, media changes, a picking step, expansion, a testing step, and freezing iPSC. It should be noted that, during the testing step, karyotype being tested may come from a frozen vial in addition to a fresh plate.

Now referring to FIG. 2C, in some embodiments, a method includes, in the following order, 2D expansion, 3D expansion, media changes, adding supplementary media, dissociation, a testing step, and freezing differentiated cells (i.e., cardiac lineage cells). In should be noted that different vessels (e.g., vessels with different properties such as, size, surface coating, gas permeability, optical clarity and/or the like that may affect cell growth, differentiation, and imaging) may be used during 3D expansion, media changes, and testing step. Additionally, or alternatively, different percentage of media change may be applied, for instance, and without limitation, depending on the vessel and cells cultured/tested in it. Vessels containing fast-growing cells (e.g., iPSCs that rapidly grow and proliferate) may require a higher percentage media change such as, 80% media change, due to the quicker depletion of nutrients and accumulation of waste products of iPSCs.

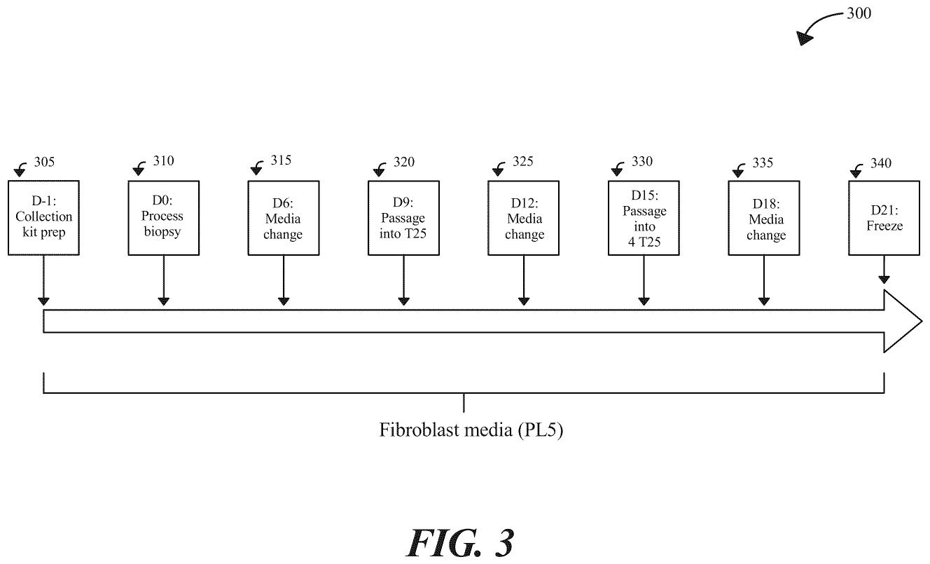

Referring now to FIG. 3, an exemplary embodiment of a method 300 of collecting, expanding, and freezing fibroblast cells is disclosed. Fibroblast cells may be collected via skin biopsy. A collection kit may be prepared on day −1 (305). A biopsy may be processed on day 0 (310). Media may be changed on day 6 (315). Cells may be transferred into a T25 flask on day 9 (320). Media may be changed on day 12 (325). Cells may be split and transferred into 4 T25 flasks on day 15 (330). Media may be changed on day 18 (335). Cells may be frozen on day 21 (340). This may be implemented without limitation, with reference to FIGS. 1-2.

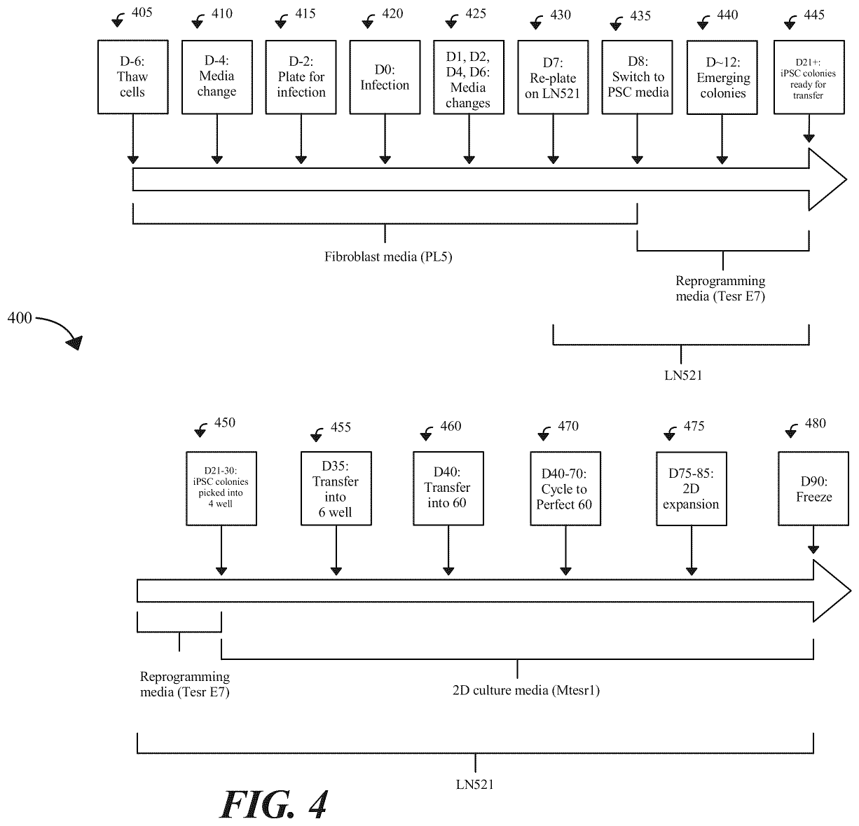

Referring now to FIG. 4, a method 400 of reprogramming cells is disclosed. In some embodiments, a method may start with a sample of frozen fibroblasts. Fibroblasts may be thawed on day −6 (405). Media may be changed on day −4 (410). Cells may be plated for infection on day −2 (415). Cells may be infected (such as with a viral vector encoding one or more reprogramming factors) on day 0 (420). Media may be changed on days 1, 2, 4, and 6 (425). Cells may be re-plated on a matrix such as LN521 (430). Cells may be switched to PSC media such as Tesr E7 on day 8 (435). iPSC colonies may emerge or may be detected on about day 12 (440). iPSC colonies may be ready for transfer on at least day 21 (445). iPSC colonies may be picked into 4 wells on day 21-30 (450). Cells may be transferred into 6 wells on day 35 (455). Cells may be transferred into a 60 mm plate on day 40 (460). Cells may be cycled to perfect 60 from day 40 to 70 (470). Cells may be expanded via 2D expansion from day 75-85 (475). Cells may be frozen on day 90 (480). This may be implemented without limitation, with reference to FIGS. 1-3.

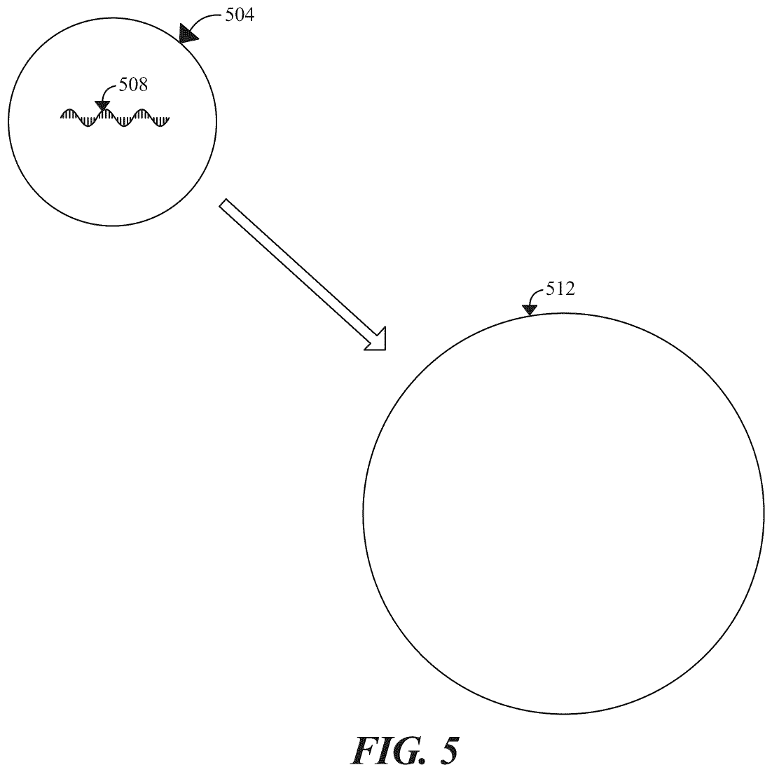

Referring now to FIG. 5, a polynucleotide 508, such as a polynucleotide encoding one or more reprogramming factors, may be delivered to a cell 512, such as a fibroblast, via a viral vector. A polynucleotide may be RNA, such as mRNA. A viral vector may include a viral particle 504. This may be implemented without limitation, with reference to FIGS. 1-4.

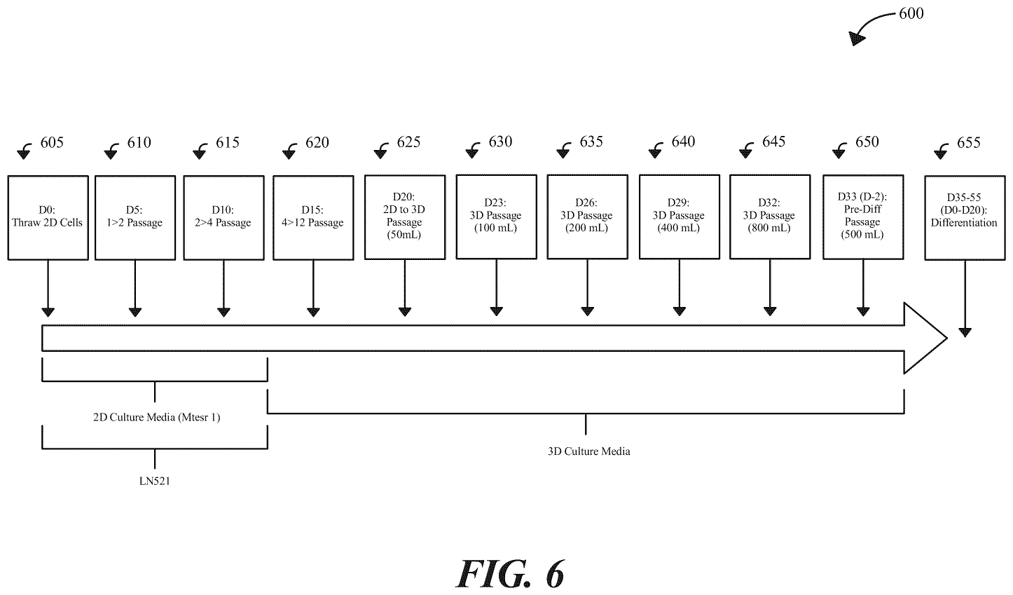

Referring now to FIG. 6, an exemplary method 600 of expanding iPSCs for differentiation is illustrated. iPSCs generated using method 400 may be thawed on day 0 (605). iPSCs may be placed in 2D culture media and 2D expanded from 1 plate to 2 plates on day 5 (610). iPSCs may be expanded from 2 plates to 4 plates on day 10 (615). iPSCs may be expanded from 4 plates to 12 plates on day 15 (620). iPSCs may then be passaged into a 50 mL vessel for 3D expansion on day 20 (625). iPSCs may be passaged into a 100 mL vessel for 3D expansion on day 23 (630). iPSCs may be passaged into a 200 mL vessel for 3D expansion on day 26 (635). iPSCs may be passaged into a 400 mL vessel for 3D expansion on day 29 (640). iPSCs may be passaged into an 800 mL vessel for 3D expansion on day 32 (645). Each passage of plurality of iPSCs may include three-day cycle of media change (i.e., 3D culture media) as described above with reference to FIG. 1. iPSCs may be passaged (i.e., pre-diff passage) into a 500 mL on day 33 (650) before iPSCs differentiation (655) from day 35 to day 55. This may be implemented without limitation, with reference to FIGS. 1-5.

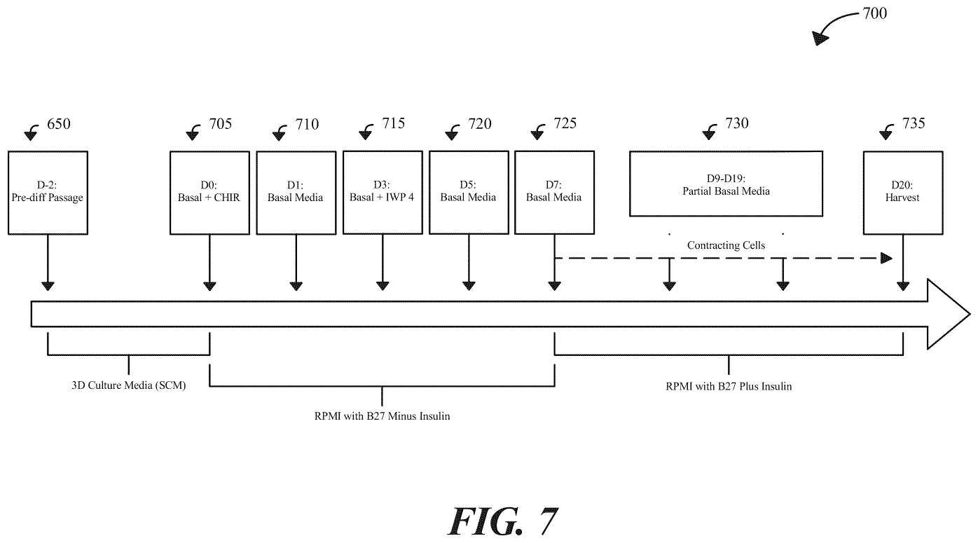

Referring now to FIG. 7, an exemplary method 700 of differentiating iPSCs into the plurality of cardiac lineage cells is illustrated. Pre-diff passage (650) may transfer iPSCs into a differentiation media for iPSCs differentiation on day 0 to day 7 (705-725), wherein the differentiation media may include RPMI with B27−/B25+ supplement (i.e., basal media). A 100% media change may be performed each day from day 0 to day 7. Supplementary media such as first inhibitor CHIR may be added to the differentiation media at step 705 on day 0. Supplementary media such as second inhibitor IWP 4 may be added to the differentiation media at step 715 on day 3. Cell contracting (730) may be performed from day 9 to day 19 on iPSCs. Contracting iPSCs may include a media change (60%) of the differentiation media to allow maturation of iPSCs. Plurality of cardiac lineage cells produced from iPSCs may be harvest on day 20 (735). This may be implemented without limitation, with reference to FIGS. 1-6.

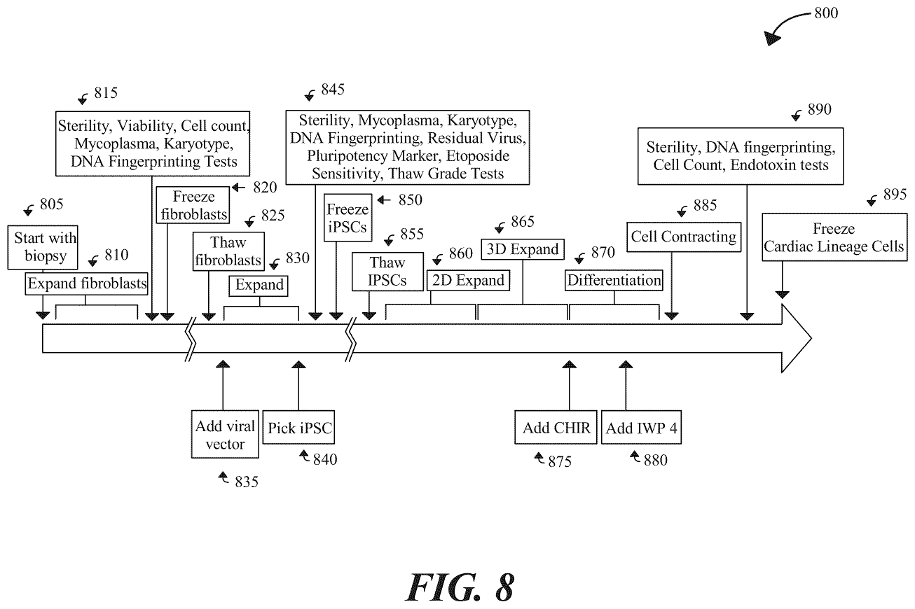

Referring now to FIG. 8, an exemplary method 800 of reprogramming cells is disclosed. Starting material may include a skin biopsy (805). Fibroblasts may be expanded (810). Sterility, viability, cell count, mycoplasma, karyotype, and DNA fingerprinting tests may be done (815). Fibroblasts may be frozen (820). Fibroblasts may be thawed (825). Cells may be expanded (830). A viral vector may be added (835). iPSCs may be picked (840). Sterility, mycoplasma, karyotype, DNA fingerprinting, residual virus, pluripotency marker, etoposide sensitivity, and thaw grade tests may be done (845). iPSCs may be frozen (850). iPSCs may be thawed (855). iPSCs may undergo a 2D expansion process (860). iPSCs may undergo a 3D expansion process (865). iPSCs may be differentiated into plurality of cardiac lineage cells (870). During iPSCs differentiation 870, CHIR may be added into differentiation media at an initial time (875), and IWP 4 may be added into differentiation media at a subsequent time (880). Cells may be contracted (885). Sterility, DNA fingerprinting, cell count, endotoxin tests may be performed (890). Plurality of cardiac lineage cells may be frozen (895). This may be implemented without limitation, with reference to FIGS. 1-7.

As used herein, the terms “administer,” “administering,” “administration,” or the like refer to the placement of a composition into a subject by any method. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

As used herein, “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” will vary depending on the composition, the condition and its severity, the manner of administration, and the age of the subject to be treated.

As used herein, “treating” or “treatment” means the treatment of a disease or condition of interest in a subject having the disease or condition of interest, and includes: (i) preventing the disease or condition from occurring in the subject, in particular, when such subject is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition, i.e., relieving pain without addressing the underlying disease or condition.

Now referring to FIG. 9, an exemplary embodiment of a system 900 for automating a method of manufacturing autologous cardiac lineage cells is illustrated. System 900 may include a computing device 904. Computing device 904 may include any computing device as described in this disclosure, including without limitation a microcontroller, microprocessor, digital signal processor (DSP) and/or system on a chip (SoC) as described in this disclosure. Computing device 904 may include, be included in, and/or communicate with a mobile device such as a mobile telephone or smartphone. Computing device 904 may include a single computing device operating independently, or may include two or more computing device operating in concert, in parallel, sequentially or the like; two or more computing devices may be included together in a single computing device or in two or more computing devices. Computing device 904 may interface or communicate with one or more additional devices as described below in further detail via a network interface device. Network interface device may be utilized for connecting a computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 904 may include but is not limited to, for example, a computing device or cluster of computing devices in a first location and a second computing device or cluster of computing devices in a second location. Computing device 904 may include one or more computing devices dedicated to data storage, security, distribution of traffic for load balancing, and the like. Computing device 904 may distribute one or more computing tasks as described below across a plurality of computing devices of computing device, which may operate in parallel, in series, redundantly, or in any other manner used for distribution of tasks or memory between computing devices. Computing device 904 may be implemented using a “shared nothing” architecture in which data is cached at the worker, in an embodiment, this may enable scalability of system 900 and/or computing device.

With continued reference to FIG. 1, computing device 904 may be designed and/or configured to perform any method, method step, or sequence of method steps in any embodiment described in this disclosure, in any order and with any degree of repetition. For instance, computing device 904 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 904 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, system 900 may include an automated blood culture system 908, a PCR system 912, and a flow cytometry system 916. In some embodiments, a computing device 904 includes a memory 920 and a processor 924. In some embodiments, an automated blood culture system 908 includes a CO2 sensor 928. In some embodiments, a PCR system 912 includes a polynucleotide detection system 932. In some embodiments, a flow cytometry system 916 includes an optical sensor 736. In some embodiments, a feature depicted in FIG. 7 may be implemented as described with reference to FIG. 1.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

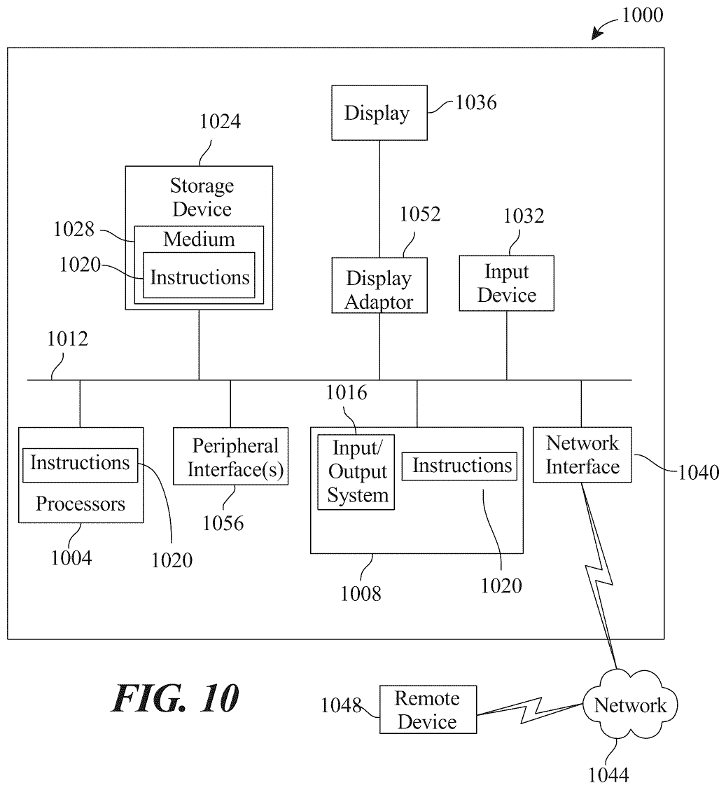

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of manufacturing autologous cardiac lineage cells, the method comprising:
receiving a patient-specific sample from a subject;
producing a plurality of fibroblast cells as a function of the patient-specific sample, wherein producing the plurality of fibroblast cells comprises:
transferring the patient-specific sample to a first growth media;
generating a plurality of induced pluripotent stem cells (iPSCs) as a function of the plurality of fibroblast cells, wherein generating the plurality of iPSCs comprises:
identifying a plurality of confluent iPSCs from the plurality of iPSCs, wherein identifying the plurality of confluent iPSCs from the plurality of iPSCs comprises:
subjecting each iPSC of the plurality of iPSCs to a quality test, wherein the quality test is an etoposide sensitivity test; and
selecting a confluent iPSC as a function of the quality test; and
differentiating the plurality of iPSCs into a plurality of cardiac lineage cells comprising cardiomyocytes, wherein differentiating the plurality of iPSCs into the plurality of cardiac lineage cells comprises:
performing a two-dimensional (2D) expansion process on the plurality of confluent iPSCs;
performing a three-dimensional (3D) expansion process on the 2D-expanded plurality of confluent iPSCs;
differentiating the 3D-expanded plurality of confluent iPSCs into autologous cardiac lineage cells; and
separating the plurality of cardiac lineage cells into a plurality of discrete cellular objects using a dissociation process, wherein the dissociation process creates a single-cell suspension from the discrete cellular objects.

2. The method of claim 1, wherein receiving the patient-specific sample comprises receiving the patient-specific sample via a skin biopsy.

3. The method of claim 1, wherein generating the plurality of iPSCs comprises:
expanding the plurality of iPSCs, wherein expanding the plurality of iPSCs comprises:
transferring the plurality of iPSCs to a vessel comprising an extracellular protein matrix.

4. The method of claim 1, wherein generating the plurality of iPSCs comprises reprogramming the plurality of fibroblast cells.

5. The method of claim 4, wherein reprogramming the plurality of fibroblast cells comprises delivering a polynucleotide encoding a reprogramming factor to the plurality of fibroblast cells via a viral vector.

6. The method of claim 5, wherein the polynucleotide encoding the reprogramming factor is an RNA.

7. (canceled)

8. The method of claim 5, wherein the viral vector comprises a Sendai viral vector.

9. The method of claim 4, wherein reprogramming the plurality of fibroblast cells comprises transferring the plurality of fibroblast cells to a reprogramming media.

10. The method of claim 1, wherein identifying the plurality of confluent iPSCs from the plurality of iPSCs comprises expanding the plurality of iPSCs.

11. (canceled)

12. The method of claim 1, wherein differentiating the plurality of iPSCs into the plurality of cardiac lineage cells comprises transferring the plurality of confluent iPSCs to a differentiation media.

13. The method of claim 12, wherein the differentiation media comprises a basal media containing a Roswell Park Memorial Institute medium (RPMI) with an optimized serum-free supplement.

14. The method of claim 12, wherein transferring the plurality of confluent iPSCs to a differentiation media comprises:
adding a supplementary media into the differentiation media, wherein the supplementary media is configured to modulate a Wnt signaling pathway.

15. (canceled)

16. The method of claim 14 wherein the supplementary media comprises a second inhibitor containing IWP 4.

17. The method of claim 1, wherein differentiating the plurality of iPSCs into the plurality of cardiac lineage cells further comprises washing the plurality of cardiac lineage cells with Dulbecco's phosphate-buffered saline (DPBS).

18. (canceled)

19. The method of claim 1, wherein the method further comprises subjecting the plurality of cardiac lineage cells to a differentiation completion test.

20. The method of claim 1, wherein the method further comprises administering a therapeutically effective amount of the plurality of cardiac lineage cells to the subject.

21. (canceled)

22. The method of claim 1, wherein separating the plurality of cardiac lineage cells into a plurality of discrete cellular objects using a dissociation process comprises mechanical dissociation.

23. The method of claim 1, wherein the iPSC's undergo a quality test, wherein the quality test is a mycoplasma test, wherein the mycoplasma test reveals if mycoplasma are present in the iPSC's.

24. The method of claim 1, wherein the iPSC's undergo a karyotype test, wherein the karyotype test measures genomic integrity of the iPSC's.

25. The method of claim 1, wherein the iPSC's undergo a DNA fingerprinting test, wherein the DNA fingerprinting test measures a genotype of the iPSC's and compares it to a genotype of a parent fibroblast sample.

26. The method of claim 1, wherein the iPSC's undergo a residual virus test, wherein the residual virus test may measure an amount of viral polynucleotide in the iPSC's.
